(19) United States
(12) Patent Application Publication
(10) Pub. No.: US 2022/0167901 A1
(43) Pub. Date: Jun. 2, 2022

(54) NEURAL RECORDING INTERFACE WITH HYBRID INTEGRATION OF NEURAL PROBE AND INTEGRATED CIRCUIT

(71) Applicant: The Regents of the University of Michigan, Ann Arbor, MI (US)

(72) Inventors: Sung-Yun PARK, Ann Arbor, MI (US); Kyounghwan NA, Sunnyvale, CA (US); Euisik YOON, Ypsilanti, MI (US)

(21) Appl. No.: 17/540,185

(22) Filed: Dec. 1, 2021

Related U.S. Application Data
(60) Provisional application No. 63/119,899, filed on Dec. 1, 2020.

(51) Int. Cl. A61B 5/293; H01L 23/00; A61B 5/273; A61B 5/304

(52) U.S. Cl. CPC A61B 5/293; H03F 3/45475; H01L 24/81; H01L 24/83; H01L 24/92; H01L 24/13; H01L 24/11; A61B 5/273; A61B 5/304; H01L 2224/11462; H01L 2224/13144; H01L 2224/13155; H01L 2224/73204; H01L 2224/8112; H01L 2224/83851; H01L 2224/92125; H01L 24/73

(57) ABSTRACT

A neural recording probe and interface, along with a method of assembly, with the neural recording probe being minimally invasive and having high-density, multi-channel microelectrodes. In one example, the neural probe includes a plurality of bumps projecting from a bottom surface of a terminal body. Each bump is electrically connected to a corresponding one of a plurality of electrodes. The plurality of bumps is bonded to a respective one of a plurality of area pads of the integrated circuit with an anisotropic conductive film such that each of the plurality of electrodes of the neural probe is electrically connected to a respective one of the active circuits of the integrated circuit.

### STATEMENT REGARDING FEDERALLY SPONSORED RESEARCH/DEVELOPMENT

This invention was made with government support under OISE1545858 awarded by the National Science Foundation. The government has certain rights in the invention.

### TECHNICAL FIELD

The present disclosure relates generally to neural recording systems and, more particularly, to a neural recording interface between a neural probe and a neural recording front-end integrated circuit.

### BACKGROUND

Simultaneous and parallel neural activity monitoring from high-density, multi-channel microelectrodes is a key requirement for understanding fundamental neural circuits and their functional connectivity in the brain. Silicon-based microelectrodes have been steadily advanced over the past few decades in order to meet such a requirement. Dense arrays of electrodes have been demonstrated that provide hundreds to thousands of simultaneous and parallel recording capabilities. For example, recently, a silicon microelectrode array has integrated 200 electrodes (9×9 μm² each) with a 11 μm inter-electrode pitch in a shank having a width of 50 μm by adopting an advanced electron-beam lithography technique (in four (4) shanks, a total of 1,000 electrodes were integrated). This design with multi-shank electrodes has been utilized to further increase the number of microelectrodes up to 4,488.

In addition, submicron CMOS processes where 6-12 metal layers are feasible can accommodate a large number of microelectrodes on a narrow probe shank. For instance, a neural probe having 1,028 channels/electrodes with multiplexing units for electrical depth control in four (4) 170 μm-wide and 4 mm-long shanks has been fabricated in a 0.5 μm CMOS process to realize 188 microelectrodes on a 40 μm-wide, 4 mm-long shank. The CMOS circuit integration has also been utilized to improve signal quality, realizing in situ signal buffering. In one example, 52 simultaneously working active pixels comprising, in part, a low noise amplifier, out of 455 active pixels have been demonstrated where the electrodes and initial amplification circuits are monolithically integrated on a 10 mm-long shank. This design has been repeatedly upgraded to provide higher channel/electrode counts that are simultaneously accessible. More importantly, the CMOS process that allows monolithic integration where the signal conditioning front-end integrated circuits and silicon-based electrodes are fabricated in the same silicon substrate completely eliminates the interconnection issue between a probe and integrated circuit, realizing each assembly for backend at high yield.

In addition to the strong demand for high-density recordings, another important consideration is the longevity of reliable recording, especially in chronic behaving animal studies. Exploration of highly flexible neural probes has recently excited the neuroscience community because of its potential for long-term stable recordings. Due to the structural and mechanical differences between silicon-based neural probes and neurons in the brain, the implanted neural probes, and shanks thereof, in particular, can lead to disruption of the native tissues that induce immune response and negatively impact stable interrogation of physiological activities over time. Research aimed to replace conventional silicon probes has explored a way to reduce stiffness by optimizing device geometry or using more flexible materials, including mesh probes that achieve tissue-like flexibility.

However, flexible materials used for neural probes, such as SU-8 or polyimide, are not compatible with the standing CMOS processes and require hybrid integration (as opposed to monolithic integration) between non-silicon-based neural probes and silicon-based CMOS front-end integrated circuits, which may require a special assembly technique. In fact, the size of the neural probe backend for interconnection becomes relatively large when scaling up the number of probe electrodes/channels. For example, the backend may occupy an estimated area as large 12,467 mm² (137 mm×91 mm) when a standard wire-bonding process is employed in a planar backend to accommodate over 1,000 channel/electrode connections between the probe and commercial recording front-end integrated circuits. This is roughly 1,000-times larger than the size of the probe itself.

Modular, expandable approaches were able to mitigate this issue by stacking modules in three-dimensional configuration. More specifically, in one example, sixteen (16) 64-channel modules each consisting of a 64-channel polymer probe and a 64-channel commercial integrated circuit formed a 1,024-channel module stacked via mezzanine connectors for long-term recording. The backend footprint of such a design is relatively small (estimated around 125 mm²) for 64 channels. However, the interconnection volume is still bulky and not scalable to accommodate additional stacking of unit probes in a headstage for future massive-parallel recording systems.

Accordingly, there is a need for a compact and reliable hybrid integration of a neural recording interface that can achieve high-density and longevity targets, while also minimizing and/or eliminating one or more of the above-identified deficiencies in conventional neural recording interfaces.

### SUMMARY

In accordance with one embodiment, there is provided a neural recording interface, comprising a neural probe. The neural probe includes an elongate cable having a first end and a second end, an implantable portion at the first end of the cable and including plurality of electrodes, and a terminal at the second end of the cable having a body with a top surface and a bottom surface opposite the top surface. The neural probe includes a plurality of bumps projecting from the bottom surface of the terminal body. Each bump is electrically connected to a corresponding one of the plurality of electrodes. The neural recording interface further includes a recording integrated circuit having a plurality of area pads and a plurality of active circuits each disposed under a respective one of the plurality of area pads. Each of the plurality of bumps projecting from the bottom surface of the probe terminal body is bonded to a respective one of the plurality of area pads of the integrated circuit with an anisotropic conductive film such that each of the plurality of electrodes of the neural probe is electrically connected to a respective one of the active circuits of the integrated circuit.

In accordance with another embodiment, there is provided a neural recording integrated circuit configured for use with a neural probe. The neural recording integrated circuit includes a plurality of active pixel circuits. Each active pixel circuit includes an amplifier having an inverting input, a non-inverting input, and an output. The active pixel circuit includes a coupling capacitor electrically connected to the inverting input of the amplifier and configured to electrically couple the amplifier with a respective electrode of the neural probe when the integrated circuit is electrically coupled with the neural probe, a feedback capacitor electrically connected between the inverting input and the output of the amplifier, and a resistor electrically connected in parallel with the feedback capacitor.

In accordance with another embodiment, there is a method of assembling a neural recording probe having a terminal with a plurality of bumps projecting from a bottom surface thereof, with a neural recording front-end integrated circuit having a plurality of area pads each having an active circuit buried thereunder. The method comprises the steps of depositing an anisotropic conductive film (ACF) onto area pads of the integrated circuit, aligning a terminal of the neural probe with the integrated circuit such that the bumps projecting from the bottom surface of the terminal are aligned with respective area pads of the integrated circuit, applying pressure between the terminal and the integrated circuit at a predetermined temperature, and cooling the terminal and the integrated circuit until the ACF hardens.

### BRIEF DESCRIPTION OF THE DRAWINGS

FIG. 1 is a diagrammatic view of an illustrative embodiment of a neural recording interface with a hybrid integration of a neural probe and a neural recording front-end integrated circuit;

FIG. 2 is diagrammatic illustration of an embodiment of a process for fabricating at least a portion of a neural probe;

FIG. 3 is a diagrammatic and cross-sectional view of an embodiment of a recording front-end integrated circuit;

FIG. 4 is a diagrammatic and cross-sectional view of an embodiment of a hybrid interface between a neural probe and the integrated circuit illustrated in FIG. 3;

FIG. 5 is a diagrammatic and schematic view of an illustrative embodiment of an active circuit/pixel of a recording front-end integrated circuit such as that illustrated in FIG. 3;

FIG. 6 is a diagrammatic and schematic view of another illustrative embodiment of an active circuit/pixel of a recording front-end integrated circuit such as that illustrated in FIG. 3;

FIG. 7 is a diagrammatic and schematic view of an illustrative embodiment of a low noise amplifier used in an active circuit/pixel such as that illustrated in FIG. 6;

FIG. 8 is a diagrammatic and schematic view of an illustrative embodiment of a recording front-end integrated circuit;

FIGS. 9A and 9B are diagrammatic and schematic views of different illustrative embodiments of a sampling circuit used in a successive approximation register analog-to-digital converter;

FIG. 10 a flowchart of an illustrative embodiment of a method of assembling a neural recording probe with a neural recording front-end integrated circuit;

FIG. 11 is a microphotograph of a fabricated prototype of a neural recording front-end integrated circuit;

FIG. 12 is a photograph of a neural recording interface with hybrid integration of a flexible neural probe and a recording integrated circuit assembled using the method illustrated in FIG. 10;

FIGS. 13A-17B are charts and graphs showing performance characteristics of components of a prototype of the neural recording front-end integrated circuit illustrated in FIG. 8 obtained during benchtop testing of the prototype;

FIG. 18 is a table showing a summary of the performance of a neural recording interface with hybrid integration of a neural probe with a prototype of the neural recording front-end integrated circuit illustrated in FIG. 8 obtained during benchtop testing of the interface and a performance comparison with other known interfaces; and

FIGS. 19 and 20 illustrate recorded signals from different channels/electrodes of a neural probe obtained during in vivo testing of a neural recording interface with hybrid integration of a neural probe with a prototype of the neural recording front-end integrated circuit illustrated in FIG. 8.

### DESCRIPTION OF EMBODIMENTS

One aspect of the present disclosure relates to a minimally invasive high-density neural recording interface for hybrid integration between a flexible neural probe (e.g., a flexible polyimide probe) and a neural recording front-end integrated circuit (e.g., a CMOS chip). In an embodiment, the neural recording interface accommodates at least 256 channels (i.e., the probe is a 256-channel probe and the integrated circuit is a 256-channel integrated circuit) and occupies a footprint or area of approximately 1.53 mm² In at least some embodiments, an integrated circuit implemented with an analog circuit-under-pad scheme with a pitch of around 75 μm, and a flip-chip bonding technique using anisotropic conductive film (ACF) to electrically connect and bond the backend of the neural probe with the integrated are employed to implement the interface.

Another aspect of the disclosure relates specifically to the front-end integrated circuit. In an embodiment, the integrated circuit includes, at least in part: an active pixel comprising an amplifier (e.g., a low noise amplifier); a programmable gain amplifier (PGA); an analog-to-digital converter (ADC); and a series-to-parallel interface (SPI). To achieve high circuit performance needed for recording neural signals in a small area (e.g., 0.0056 mm², which is equivalent to a 75 μm pitch), a reference-replica topology is implemented to provide a matched input impedance in the signal and reference paths of the amplifier of the pixel and to achieve low noise and high common mode rejection ratio (CMRR). In an embodiment, the integrated circuit is directly flip-chip bonded with the neural probe backend and assembled on a headstage printed circuit board (PCB).

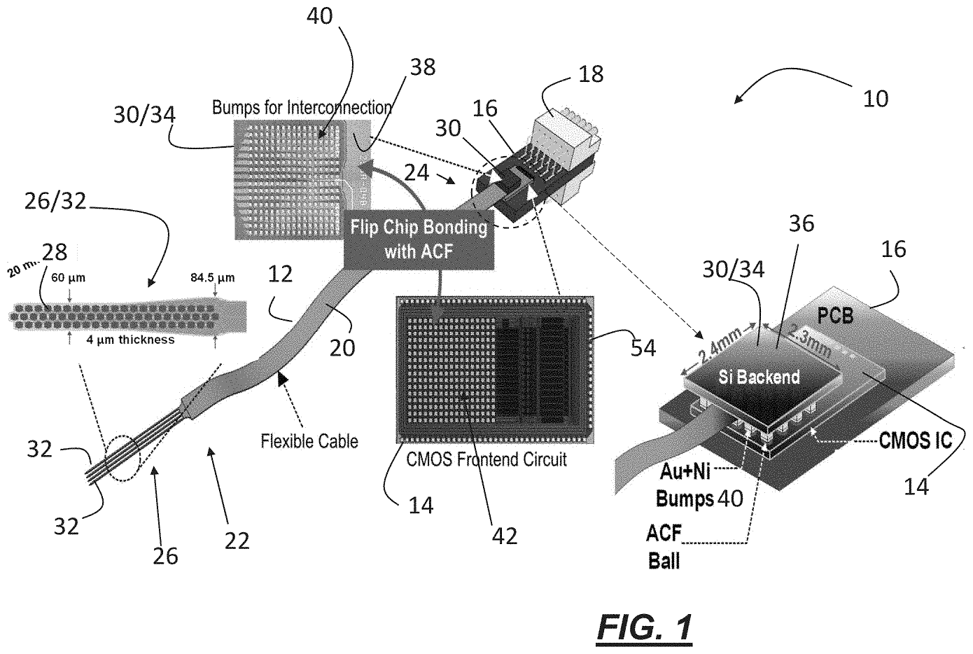

Turning now to the drawings, FIG. 1 depicts an illustrative embodiment of a neural recording interface 10 with hybrid integration of a neural probe 12 and a neural recording integrated circuit 14 that may include, for example, the neural probe 12, the integrated circuit 14 implemented on a CMOS chip, a headstage printed circuit board (PCB) 16, and a connector 18 to facilitate the connection of the integrated circuit 14 to other components of a neural recording system.

In an embodiment such as that shown in FIG. 1, the neural probe 12 comprises a multi-channel flexible probe that includes an elongate flexible cable 20 having a first end 22 and second end 24, an implantable portion 26 at the first end 22 of the cable 20 and including a plurality of electrodes 28, and a terminal or backend 30 at the second end 24 of the cable 20 configured to electrically and mechanically couple or connect the probe 12 with the integrated circuit 14.

The flexible cable 20 is configured to electrically connect the electrodes 28 at the first end 22 of the cable 20 to the terminal or backend of the probe at the second end 24 of the cable. Accordingly, the cable 20 include a plurality conductors or electrical traces (not shown) each extending the length of the cable 20 from a respective one of the electrodes 28 to the terminal 30. The flexible cable 20 may be formed of various materials. In one embodiment, the cable 20 is formed of polyimide, while in other embodiments the cable may be formed of another suitable material, for example, parylene-C or SU-8. The cable 20 may also have various dimensions. In one embodiment, the cable has a length of 20-25 mm and a width of 2-3 mm, while in other embodiments, the cable 20 may have a different length and/or width.

The probe 12, and the implantable portion 26 thereof, in particular, may include any number of electrodes, which may be formed of platinum and/or any other suitable material. Each of the plurality of electrodes 28 may comprise either a recording electrode or a reference electrode, and the probe 12 will include at least one recording electrode and at least one reference electrode. The number of recording electrodes dictates the number of channels the probe 12 includes. In one illustrative embodiment, the probe 12 includes 256 recording electrodes, and thus, the probe 12 comprises a 256-channel probe. In such an embodiment, the probe 12 will further include one or more reference electrodes. In one particular implementation, the probe 12 includes 16 reference electrodes, and thus, the total number of electrodes 28 the probe 12 has is 272. It will be appreciated, however, that the present disclosure is not limited to any particular number of recording electrodes and/or reference electrodes. Each electrode 28 of the probe 12, regardless of whether it is a recording electrode or references electrode, is electrically connected to a respective one of the plurality of electrical conductors of the cable 20 extending along the length of the cable 20 from the electrode 28 to the terminal 32.

The implantable portion 26 of the probe 12 may include a number of implantable shanks 32 each carrying one or more of the electrodes 28. For example, in an embodiment wherein the probe 12 has 256 electrodes, the probe 12 may include four (4) shanks 32 each having 64 electrodes incorporated therein. The electrodes 28 on a particular shank 32 may be arranged in any number of ways. One illustrative way is that depicted in FIG. 1 wherein the 64 electrodes 28 are arranged in three rows with the electrodes 28 in a given row being staggered or offset from the electrodes 28 in one or more immediately adjacent rows so as to form a polytrode. However, other suitable electrode arrangements may certainly be used instead. The shanks 32 may have a variety of dimensions. In the embodiment illustrated in FIG. 1, for example, each of the shanks 32 has a maximum width of 84.5 μm, which tapers down to 60 μm at a distance of ˜220 gm from the tip of the shank 32, and length of 6 mm. It will be appreciated, however, that other suitable dimensions are certainly possible.

The dense arrangement of the electrodes 28 in a small geometric area offers high spatial resolution and tetrode-like redundancies, which may improve spike sorting accuracy. However, the small geographic area also increases the impedance of the electrodes 28, resulting in a low signal-to-noise ratio (SNR). Additional plating of PEDOT-PSS and Pt-black can be applied to lower the electrode impedance, but this typically requires plating of individual sites one-by-one. Rather than plating the electrodes, in one embodiment, the impedance of the electrodes 28 is decreased by roughening the polymer substrate before the electrodes 28 are deposited. This process effectively increases the surface area of the electrodes 28 and achieves a low impedance in a small geometric electrode at high-throughput from wafer-level processes. In testing, it was found that the impedance of the roughened electrodes was three (3) times less than that of flat electrodes.

In an embodiment, the terminal or backend 30 of the probe 12 has a rigid body 34 with a top surface 36 and bottom surface 38. In embodiment, the body 34 of the terminal 30 is formed of silicon, but other suitable materials may be used instead. As shown in FIG. 1, the terminal 30 includes a plurality of bumps 40 projecting outwardly from the bottom surface 38 of the terminal body 34. Each bump 40 is electrically connected to a respective one of the electrical conductors of the cable 20 such that each bump 40 is electrically connected to a respective one of the plurality of the probe electrodes 28. Accordingly, in an embodiment, the terminal 30 has the same number of bumps 40 as the probe 12 has electrodes 28. For example, in the embodiment described above wherein the probe 12 has 272 electrodes (e.g., 256 recording electrodes and 16 reference electrodes), the terminal 30 may have 272 bumps. In such an embodiment, the bumps 40 may be arranged in a 16×17 array with a 75 μm pitch.

The bumps 40 may be formed of a variety of materials. In an embodiment, the bumps are formed of nickel and coated with gold. And the bumps 40 may be of any size that is suitable for use for the purposes described herein. In an embodiment, however, the bumps 40 may have a height of 10-25 μm, and preferably, 15-20 μm. In one illustrative embodiment, the bumps 40 are formed by electroplating nickel having a height of 16-20 μm and then coating the nickel with 100 nm-thick sputtered gold to prevent or at least limit oxidation.

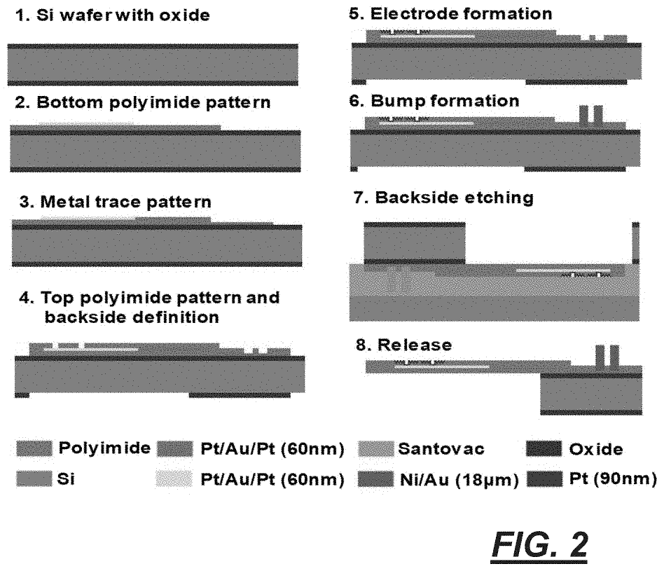

The probe 12 described above may be fabricated in a number of ways. One such embodiment is illustrated in FIG. 2. The process illustrated in FIG. 2 begins with a silicon wafer with a 2 μm-thick thermally grown SiO₂ layer and a Cr/Au/Cr sacrificial layer on top of that. A first polyimide layer is then spun and cured on the wafer. Fine feature metal traces (Pt/Au/Pt, 10/40/10 nm) are then patterned on probe shanks by lift-off Another lithography step is performed to define large-feature metal traces (Pt/Au/Pt, 25/250/25 nm) on the cable 20 and backend 30 after etching polyimide on the backend 30. A second polyimide layer is then spun and fully cured, followed by plasma patterning. Platinum electrodes are the deposited and patterned on top of a roughened surface. The nickel bumps 40 are formed through electroplating and then covered with gold for protection. The wafer is then mounted on a carrier wafer using a bonding material such as, for example, Santovac, and silicon is etched from the backside by deep reactive ion etching (DRIE) with silicon dioxide as a mask until the etch stop layer (silicon dioxide) is exposed. The remaining silicon under the shanks of the probe is separated by sacrificial layer (Cr/Au/Cr) etching in Cr etchant, following by silicon oxide removal in BHF. Finally, the shanks are released in acetone by removing the bonding material.

Turning now to the recording front-end integrated circuit 14, in an embodiment, the integrated circuit 14 is an analog CMOS circuit implemented on a CMOS chip. Conventionally, perimeter pads of an integrated circuit are used to connect electrodes of a neural probe with a recording front-end integrated circuit. However, such a connection arrangement limits the ultrahigh density hybrid integration of the probe and integrated circuit. For example, in an instance wherein the probe comprises a 256-channel probe, and assuming a typical pad pitch of 80 μm, the perimeter connection arrangement will make the integrated circuit chip size larger than 10 mm in length when the pads are placed in both the top and bottom of the chip-making die, which is much larger than desired for a neural recording application. As will be described below, for the integrated circuit 14 of the present disclosure, area pads rather than perimeter pads are used for the interconnection between the bumps 40 of the probe backend 30 (and thus, the probe electrodes 28) and the integrated circuit 14 in order to reduce the area allocated for the interconnection between the probe 12 and the integrated circuit 14.

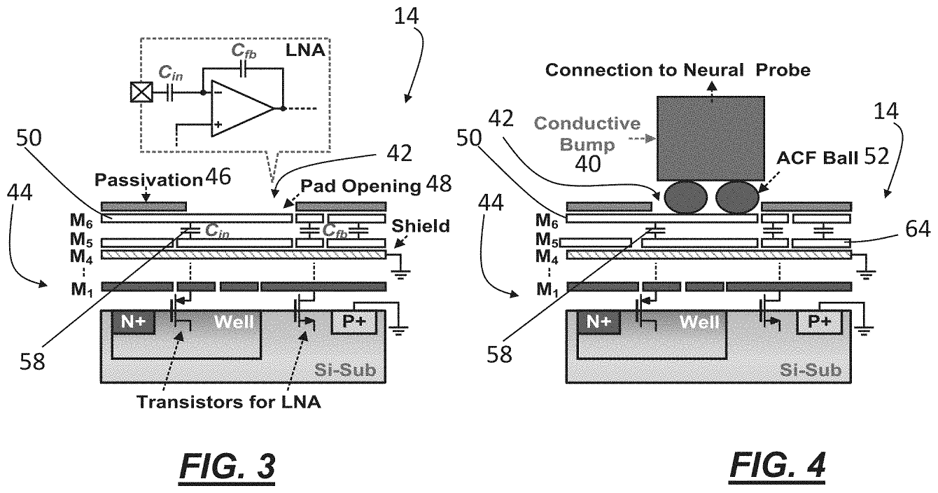

As shown in FIGS. 1 and 3, the integrated circuit comprises a plurality of area pads 42 and a plurality of active circuits 44 (shown in FIG. 3) each disposed or buried under a respective one of the plurality of area pads 42 such that each active circuit 44 is implemented using an analog circuit-under-pad scheme or technique. Placing the active circuits 44 under the area pads 42 is feasible because the mechanical stress applied to the components of the active circuits 44 resulting from an ACF bonding process/technique described elsewhere herein used to electrically connect and bond backend 30 of the probe 12 with or to the integrated circuit 14 is significantly less than the stress that would be applied using a conventional wire-bonding process/technique (i.e., estimated to be approximately ten (10) times less, i.e., in the range of 100 MPa).

The integrated circuit 14 may have any number of area pads 42 having active circuits 44 thereunder. In an embodiment, the integrated circuit 14 has at the same number of area pads 42 and active circuits 44 as the probe terminal 30 has bumps 40. So, in an embodiment such as that described above wherein the terminal has 272 bumps, the integrated circuit may have 272 area pads and corresponding buried active circuits 44. In an embodiment, the integrated circuit 14 has pad pitch of 75 μm, though the present disclosure is not limited to any particular pitch value. Further, in at least some embodiments, each channel of the integrated circuit 14, which comprises both the pad 42 and the active circuit 44 thereunder, consumes an area of approximately 0.0117-0.0175 mm², which is far smaller than most, if not all, conventional integrated circuits (e.g., area of around 0.48 mm²), with each active circuit 44 consuming approximately 75 μm². As shown in FIG. 3, in an embodiment wherein the integrated circuit 14 includes a passivation layer 46 as a top surface of the circuit 14, the circuit 14 includes a plurality of pad openings 48 that are defined by a gap or void in the passivation layer 46 and that expose and provide access to, for example, a top metal layer 50 of the integrated circuit 14 that, in some embodiments, forms part of the active circuit 44 buried under the pad 42.

In any event, some or all of the pads 42 of the integrated circuit 14 are configured to be electrically connected to and bonded with a respective bump 40 of the terminal 30 of the probe 12, such that the terminal 30 of the probe 12 is configured to be flip-chip bonded to the integrated circuit 14. In an embodiment such as that illustrated in FIGS. 1 and 4, and as will be described in greater detail below, one or more spheres or balls 52 of ACF is/are used to bond the individual bumps 40 on the bottom surface 38 of the backend/terminal 30 to the individual pads 42 of the integrated circuit 14. When the bumps 40 and pads 42 are bonded together, the active circuits 44 under those pads 42 are each electrically connected to a respective bump 40, and thus, to the electrode 28 of the probe 12 to which that bump 40 is electrically connected.

In addition to the area pads 42 configured for bonding with the bumps 40 of the probe terminal 30 to electrically connect electrodes 28 of the probe 12 with active circuits 44 of the integrated circuit 14, the integrated circuit 14 further includes one or more other pads 54, for example, perimeter pads, configured to be electrically connected to other components of the recording system. For example, the integrated circuit 14 may include pads 54 (shown in FIG. 1) electrically connected (e.g., wire-bonded) to, for example, the headstage PCB of the recording interface 10 for one or more power supplies (e.g., 1.2, 1.8, and 3.3V power supplies) and ground, input for control and setting parameters of components of the integrated circuit 14 (e.g., a data serializer), and outputs for data transfer to other components of the recording system, for example, a host or a workstation.

In an embodiment, some or all of the active circuits 44 buried under the area pads 42 of the integrated circuit 14 comprise active pixels (or active pixel circuits) that are each configured to be electrically connected or coupled to a respective electrode 28 of the probe 12. Each of the active pixels 44 will comprise either a recording pixel (designated herein by reference number 44a) or a reference pixel 44b (designated herein by reference number 44b), with each of the recording pixels 44a being configured to be electrically coupled to a respective recording electrode of the probe 12, and each reference pixel 44b being configured to be electrically coupled to a respective reference electrode of the probe 12. In an embodiment, the integrated circuit 14 includes one or more recording pixels 44a and one or more reference pixels 44b. For example, in an embodiment such as that described above wherein the probe 12 has 256 recording electrodes and 16 reference electrodes, the integrated circuit 14 may include 256 recording pixels 44a and 16 reference pixels 44b.

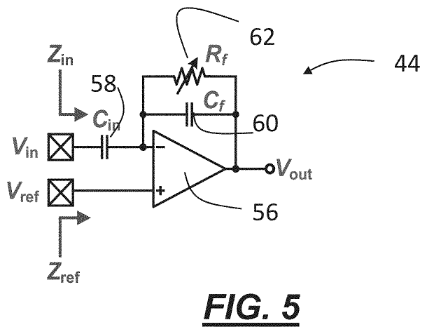

Regardless of whether an active pixel 44 is a recording pixel or a reference pixel, in at least some embodiments, the composition and structure of all of the active pixels 44 is exactly the same. More particularly, in an embodiment such as that illustrated in FIG. 5, each active pixel 44 includes, at least in part, an amplifier 56, for example and without limitation, a low noise amplifier (LNA), which, in an embodiment, may comprise an operational transconductance amplifier (OTA). While the amplifier 56 may comprise an amplifier other than an LNA, for purposes of illustration the description below will be with respect to an embodiment wherein the amplifier 56 comprises an LNA (i.e., LNA 56). In the embodiment illustrated in FIG. 4, the pixel 44 may include other components in addition to the LNA 56, for example, a coupling capacitor (or Cᵢₙ) 58, a feedback capacitor (or C_{fb} or C_{f}) 60, and a resistor (or R_{f}) 62.

In the embodiment illustrated in FIG. 5, the LNA 56 is implemented in an unbalanced single-ended, ac-coupled structure wherein the non-inverting input of the LNA 56 is electrically connected with the coupling capacitor 58 that is configured to electrically connect or couple the LNA 56 with an electrode 28 of the probe 12. In an instance wherein a particular active pixel 44 is a recording pixel, the coupling capacitor 58 is configured to electrically connect the LNA 56 with a recording electrode, whereas in an instance wherein a particular active pixel 44 is a reference pixel, the coupling capacitor 58 is configured to electrically connect the LNA 56 with a reference electrode. The coupling capacitor 58 may comprise a metal-insulator-metal (MIM) capacitor that is formed by a pair of metal layers and an insulator layer of the integrated circuit 14. For example, FIGS. 3 and 4 depict the coupling capacitor 58 being formed in part by the first or top metal layer 50 of the integrated circuit 14 and a second metal layer 64 below the first or top metal layer 50. In such an embodiment, and as shown in FIG. 4, the bumps 40 of the terminal 30 of the probe 12 are bonded to respective portions of the first or top metal layer 50, and thus, are electrically connected to the coupling capacitors 58 of respective active circuit/pixels 44. In such an implementation, additional routing from the top metal layer 50 coupled to the bumps 40 to the pixel 44 is not needed.

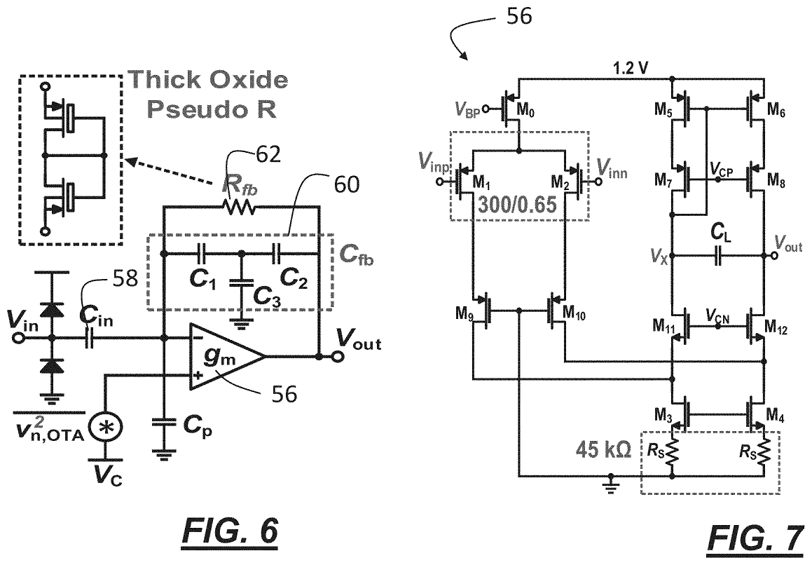

In the unbalanced single-ended structure illustrated in FIG. 5, the feedback capacitor 60 is electrically connected between the inverting input of the LNA 56 and an output of the LNA 56, and the resistor 62 is electrically connected in parallel to the feedback capacitor 60. The feedback capacitor 60 may comprise a single capacitor or may be comprised of multiple capacitors that are electrically connected to each other to form the feedback capacitor 60. For example, and with reference to FIG. 6, in a particular implementation of the LNA 56, the overall capacitance is on the order of 6,160 fF, which fits well in 5,625 μm² considering a MIM capacitance density of 2 fF/μm². The limited size of the input capacitance (Cᵢₙ) may contribute to noise multiplication due to parasitic capacitance of input transistors represented as “Cₚ” in FIG. 6. Given a value of around 615 fF. for parasitic capacitance, the noise multiplication factor can be restricted under 20% by choosing a value of 3.13 pF for Cᵢₙ. To generate a large amplifier gain of around 43 dB, the feedback capacitor 62 would need to have a capacitance as small as 25 fF. To realize such a low capacitance for the feedback capacitor 60, a T-network comprised of three separate capacitors (C₁, C₂, and C₃) is used. The input capacitance of 3.31 pF results in approximately 48 MΩ of input impedance in the LNA 56 at 1 kHz. To generate a high frequency cut-off, a load capacitor (C_{L}) having a capacitance of 2.4 pF is connected at the output of the LNA 56, referenced to an internal node of the LNA 56 to double the effective capacitance.

As it relates to the LNA 56, 1/f noise is inherent in metal oxide semiconductor devices and can be reduced by increasing the area of the devices or using circuit design techniques, such as auto-zeroing or chopper stabilization. However, these circuit techniques require additional components, for example, switches, filters, and/or servo loops, and/or a large sampling frequency. This makes it difficult to implement a compact LNA inside a limited area. In an embodiment of the LNA 56, the area of the input transistors of the LNA 56 is maximized to reduce 1/f noise.

Additionally, thermal noise is attenuated to meet an overall input-referred noise (IRN) requirement for the application. Larger spot noise for local field potentials (LFP) and smaller spot noise for extracellular action potentials (ExAPs or spikes) can be tolerated since the amplitude of an LFP (1-3 mV) is usually an order of magnitude higher than that of spikes (10-100 μV). In order to reduce the thermal noise, the LNA 56 is designed to consume a larger current than other known LNAs, for example, a current on the order of ˜7.2 μA. The thermal noise floor was projected to be <10 nV√z, equivalently <1.0 μmVᵣₘₛ IRN considering only thermal noise up to 10 kHz, which is at least three (3) to five (5) times smaller than the conventional LNAs.

FIG. 7 shows an embodiment wherein the LNA 56 is implemented as an OTA. In this implementation, most of the current is dissipated in the input transistors M₁ and M₂ to maximize the overall transconductance, and only 1/16 of the input current flows into the output branch to save the power consumption since no high slewing is required for neural recording applications. In the given area restriction, the size of input transistors M₁ and M₂ is maximized to suppress the 1/f noise. The simulated IRN from 1.0 Hz to 1 MHz is ˜4.7 μVᵣₘₛ, which is within a safe margin even if considering √2× multiplication of IRN by the reference-replica configuration or scheme described elsewhere herein. IRN is dominated by the 1/f noise. A 45 kΩ source degeneration resistor (R_{S}) may be used to limit the noise contribution from transistors M₃ and M₄.

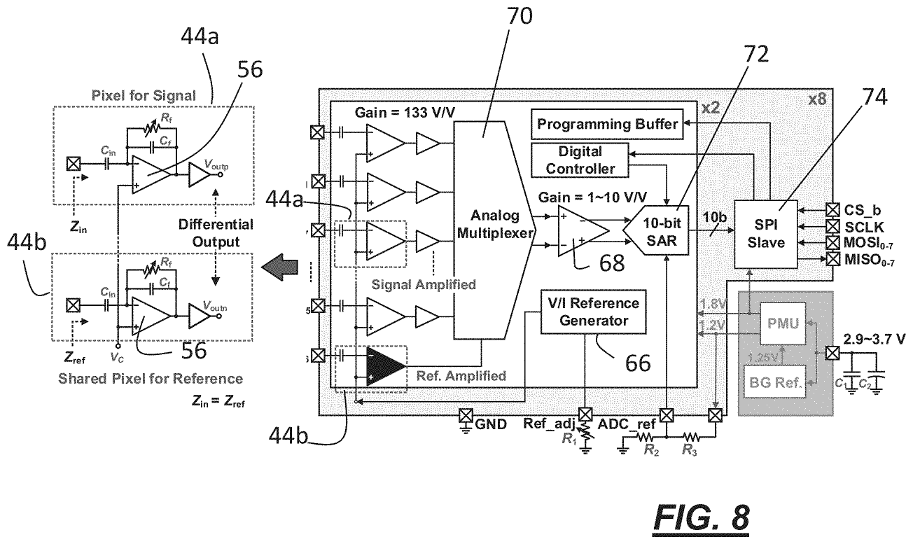

In any event, while the inverting input of the LNA 56 is electrically coupled with an electrode of the probe 12, the non-inverting input is not. Instead, the non-inverting input of the LNA 56 is electrically connected with the non-inverting input of the LNA 56 of another active pixel 44, which, in turn, may be electrically connected to a common voltage (V_{c}), such that the non-inverting inputs of the LNAs of both active pixels 44 are electrically coupled to the same common voltage. More particularly, and as illustrated in FIG. 8, the inverting input of the LNA 56 of a recording pixel 44a is configured to be electrically connected to a recording electrode, whereas the inverting input of the LNA 56 of a reference pixel 44b is configured to be electrically connected to a reference electrode. Both the non-inverting input of the LNA 56 of the recording pixel 44a and the non-inverting input of the LNA 56 of the reference pixel 44b are electrically connected to each other and a common voltage. Accordingly, and as shown in FIG. 6, the non-inverting input of the LNA 56 of each recording pixel 44a is electrically connected to both the non-inverting input of the LNA 56 of a reference pixel 44b and a common voltage. Because the non-inverting input of the recording pixel 44a is connected to the non-inverting input of the reference pixel 44b having the same design as that of the recording pixel 44a, and the non-inverting inputs of both the recording and reference pixels 44a, 44b are electrically connected to a common voltage, the input impedance (Zᵢₙ) of the signal path or inverting input of the recording pixel 44a and the input impedance (Z_{ref}) of the reference path or non-inverting input of the recording pixel 44a are the same and a relatively high common mode reduction ratio (CMRR) is achieved. Since the recording and reference pixels 44a, 44b are structurally the same and are electrically connect to each other, this scheme or topology is referred to herein as a “reference-replica” configuration, scheme, or topology.

In some embodiments, such as, for example, that illustrated in FIG. 8, a single reference pixel 44b may be shared by or assigned to multiple recording pixels 44a. In such an embodiment, the inverting input of the LNA of each recording pixel 44a is configured to be electrically connected to a respective recording electrode of the probe, and the inverting input of the LNA of the reference pixel 44b is configured to be electrically connected to a reference electrode of the probe 12. As illustrated in FIG. 8, the non-inverting inputs of the LNAs 56 of the recording pixels 44a are all electrically connected together and to the non-inverting input of the LNA 56 of the reference pixel 44b and a common voltage.

The common voltage to which both recording and reference pixels 44a, 44b are electrically connected may take a number of forms. In some embodiments, the common voltage (V_{c}) may comprise a common mode voltage (V_{cm}). In other embodiments, the common voltage may comprise an artificially generated reference voltage such as, for example, a common average reference (V_{car}) to improve signal quality. In the latter instance, the integrated circuit may include a voltage reference generator 66 as shown in FIG. 8.

In addition to the active circuits/pixels 44 described above, the integrated circuit 14 may further include one or more additional components. For example, in the embodiment illustrated in FIG. 8, the integrated circuit 14 further includes a programmable gain amplifier (PGA) 68 electrically connected either directly or indirectly via one or more other components to the outputs of one or more of the pixels 44 (e.g., the output(s) of one or more recording pixels may be electrically connected to one input of the PGA, and the output of a reference pixel may be electrically connected to another input of the PGA). In at least some embodiments, the PGA 68 may comprise an ac-coupled, fully-differential amplifier, and may provide a plurality of different gains. For example, in an embodiment, the PGA 68 may include four different gains: 0, 3, 5, and 10 dB; while in another embodiment, the four gains of the PGA may be 0, 5.6, 9, and 20 dB. Accordingly, the present disclosure is not intended to be limited to any particular number or values of the gains. In at least some embodiments, the PGA 68s comprises an OTA implemented in a two-stages to maximize the signal swing.

As shown in FIG. 8, the integrated circuit 14 may further include a multiplexer 70 having a plurality of inputs and an output. In an illustrative embodiment, the multiplexer 70 comprises an analog time-division-multiplexer (A-TDM). The A-TDM is electrically connected between the active pixels 44 and the PGA 68 such that the inputs of the A-TDM are electrically connected to the outputs of the active pixels 44 (e.g., the outputs of the LNAs 56 thereof), and the output of the A-TDM is electrically connected to an input of the PGA 68. Accordingly, in an embodiment, the output(s) of two or more recording pixels may be indirectly electrically connected to an input of the PGA 68 via the A-TDM 70, and the output of a reference pixel may be indirectly electrically connected to another input of the PGA 68 either directly or indirectly via the A-TDM 70. In an embodiment wherein the multiplexer 70 is an A-TDM, each active circuit/pixel 44 may further include a transmission gate switch that is necessary for the implementation of the A-TDM 70. In an embodiment wherein the integrated circuit 14 includes the A-TDM 70, the signals output by multiple active pixels 44 are in the time domain. The ratio of the A-TDM 70 is dependent upon the number of signals that need to be multiplexed, and thus, the number of pixels 44. For example, in an instance wherein the integrated circuit comprises 16 active recording pixels 44a, the A-TDM will have a 16:1 ratio and will multiplex all 16 signals. In one implementation wherein the A-TDM comprises a 16:1 multiplexer, the sampling rate is set at 31.25 kS/s for each channel, and so the multiplying frequency is on the order of 500 kS/s.

In order to reduce power consumption, the integrated circuit 14 may further include an analog-to-digital converter (ADC) 72 having an input and an output, with the input of the ADC 72 being electrically connected to the output of the PGA 68. In an embodiment, the ADC 72 comprises a successive approximation register (SAR). The particular resolution of the ADC 72 is dependent at least in part on the dynamic range of the broadband neural signals received from the electrodes 28 of the probe 12, which is on the order of 60 dB. In an embodiment, the SAR ADC 70 may have a resolution of 10-12 bits.

The SAR ADC 72 is one of the most widely adopted Nyquist rate ADC topologies for neural recording applications due to its low power consumption. However, SAR ADCs consume a large area, which increases exponentially as the number of bits increases. This poses a drawback of SAR ADCs when used in high-resolution applications, such as, for example, neural recording applications. In an embodiment, the SAR ADC 72 described above is a fully-differential 10-bit SAR ADC and is implemented in a smaller area by reducing the size of the capacitive digital-to-analog-converter (CDAC) of the SAR ADC 72 by approximately four (4) times compared to conventional CDACs.

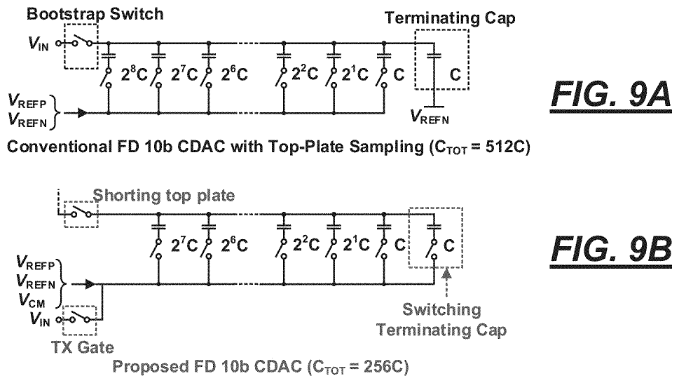

FIG. 9A illustrates an example of top-plate sampling that can reduce the capacitance of the CDAC by half through making the Most Significant Bit (MSB) decision without bit cycling. However, the top-plate sampling mandates a bootstrap switch that consumes additional power and area. To avoid using a bootstrap switch, the input is sampled at the bottom plate of the capacitors and the top plates are shorted, and then the common mode (CM) voltage (V_{CM1}) is applied for MSB decision without bit cycling, as is illustrate in FIG. 9B. This scheme or configuration also reduces the total capacitance of the CDAC by half. The penalty is a sign reversal in the input signal, which can be easily managed by SAR control logic.

After sampling and bootstrapping the input signals (vᵢₚ, vᵢₙ), the top plate voltages (vₓₚ, vₓₙ) of the differential DAC are given as Vₓₚ=−0.5 v_{id}+V_{CMI}, and vₓₙ=0.5 v_{id}+v_{CM1}, where via is the differential voltage of inputs to the ADC (vᵢₚ=0.5 v_{id}+V_{CMO}, vᵢₙ=−0.5 v_{id}+V_{CM0}) and V_{CM1} is the common mode voltage for the SAR ADC. In the implementation illustrated in FIG. 9B, by shorting the top plates before reference to V_{CM1}, a common mode mismatch between the previous stage V_{CM0} and the current one (V_{CM1}) can be eliminated. In addition to the above, in an embodiment such as that illustrated in FIG. 9B, a terminating capacitor switching scheme may be added to further reduce the size of the CDAC by, for example, another half. Since the terminating capacitor joins in the Least Significant Bit (LSB) cycling process by switching only one of the top plates, the total capacitor array size becomes half without aggravating any decision errors. However, in the course of LSB decision, it may slightly affect the decision threshold from the common mode voltage variation in the comparator, which may possibly introduce a small signal-dependent offset.

Finally, in the CDAC, a unit capacitor may comprise a MIM capacitor and may have a capacitance of ˜17 fF. The overall sampling capacitor thus becomes ˜4.48 pF for fully differential operations. All switches and active components are buried under the MIM capacitor to further limit or reduce area consumption. An additional benefit of the V_{CM}-based switching scheme described above is that it consumes ˜70% less energy in the DAC switching, even with the increased bottom plate parasitic capacitance by adding four switches. By applying the V_{CM}-based switching and terminating capacitor switching scheme/configuration, a fully differential 10-bit SAR ADC is implemented in a small area of 75×350 μm² having a power consumption of ˜9.05 μW at its full speed of 500 kS/s.

In addition to those components described above, in at least some embodiments, the integrated circuit 14 may also include a data serializer 74, for example, a series-parallel interface (SPI). As shown in FIG. 8, the SPI 74 has an input and an output, wherein the input is electrically connected to the output of the ADC 72. In an embodiment, the data serializer/SPI 74 may have a data rate between 20 kS/c and 5 MS/s, though other suitable rates or ranges of rates may certainly be used instead.

In an embodiment wherein the integrated circuit includes one or more of the PGA 68, the A-TDM 70, the ADC 72, and/or the data serializer/SPI 74, the integrated circuit 14 may include one of each of that or those components, or may include two or more of some or all of those components. The number of components the integrated circuit 14 includes is may depend, at least in part, on the number of active circuits/pixels 44 the integrated circuit 14 includes.

For example, in an embodiment wherein the integrated circuit 14 includes a relatively low number of active circuits/pixels 44, for example, 16 active recording pixels 44a and one (1) active reference pixel 44b, the integrated circuit 14 may include one of each of the PGA 68, the A-TDM 70 (i.e., a 16:1 A-TDM), the ADC 72, and/or the data serializer 74. In other embodiments, however, wherein the integrated circuit 14 includes a larger number of active circuits/pixels 44, the active circuits/pixels 44 may be divided into different groups with each group comprising a subset of the active circuits/pixels 44. In such an embodiment, each group may also include one of each of the PGA 68, the A-TDM 70, the ADC 72, and/or the data serializer 74.

For example, in an embodiment such as that described herein wherein the integrated circuit 14 includes 272 active circuits/pixels 44 (i.e., 256 active recording pixels 44a and 16 active reference pixels 44b), the active circuits/pixels 44 may be divided into 16 different groups with each group including 16 active recording pixels 44a and one (1) reference pixel 44b. In such an embodiment, the integrated circuit 14 may include PGA 68 for each group, an A-TDM 70 for each group (i.e., a 16:1 A-TDM), an ADC 72 for each group, and a data serializer 74 for each group. Accordingly, in such an embodiment, the integrated circuit 14 may include 16 PGAs 68, 16 A-TDMs 70, 16 ADCs 72, and 16 data serializers 74. In other embodiments, however, two or more groups may share one or more of these components. For example, two groups may share a single data serializer 74 such that the integrated circuit 14 would include eight (8) data serializers 74. In such an embodiment, the data serializer 74 may be implemented in a column-parallel fashion providing 256-channel simultaneous recording.

It will be appreciated in view of the description above that in an instance wherein the integrated circuit 14 includes different groups comprised of different active pixels 44, each of the active recording pixels 44a in a particular group are electrically connected to the reference pixel 44b of that group and a common voltage. That is, the non-inverting inputs of the LNAs 56 of the recording pixels 44a are all electrically connected to the non-inverting input of the reference pixel 44b, the non-inverting inputs of the other recording pixels 44a, and a common voltage.

As briefly discussed above, in addition to the integrated circuit 14 and the headstage

PCB 16 described above, the neural recording interface 10 may further include one or more connectors 18 for connecting the interface 10 to, for example, one or more other components of a larger neural recording system. In an embodiment such as that illustrated in FIG. 1, the connector 18 is mounted on the headstage PCB 16 and is electrically connected to the integrated circuit 14. While the connector 18 may comprise any suitable connector, in an illustrative embodiment, the connector 18 comprises a 12-pin connector. In other embodiments, however, other connectors may be used to reduce the area of the headstage PCB 16, for example, a mezzanine connector. Further, to stabilize the operation of the integrated circuit 14, the recording interface 10 may also include one or more decoupling capacitors assembled in the headstage PCB 16.

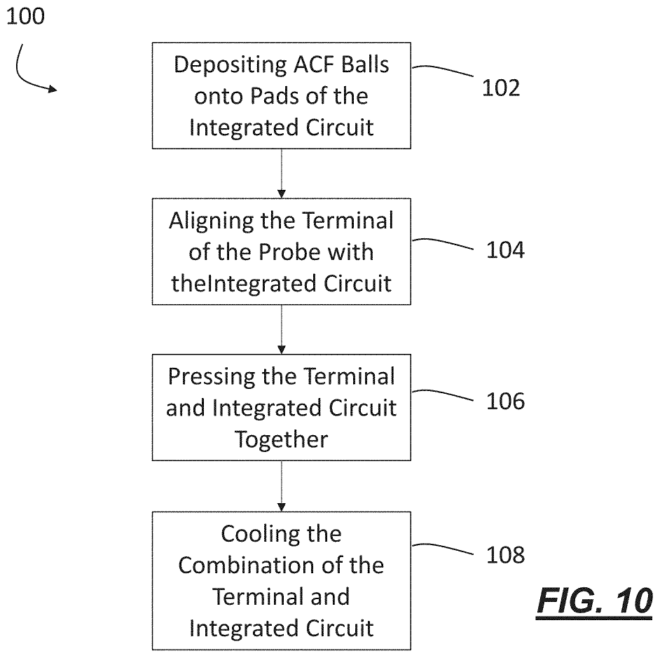

In addition to the structure of the recording interface 10 and the integrated circuit 14 described above, another aspect of the disclosure relates to a method 100 of assembling the components of the recording interface together. FIG. 10 depicts an illustrative embodiment of the method that employs a flip-chip bonding technique with ACF.

Flip-chip bonding is one of the most widely accepted technologies for high-density interconnection of integrated circuits. In conventional flip-chip bonding processes, solder balls are formed on bonding pads of an integrated circuit by either electroplating, solder ball placement, or stencil printing on top of under-bump-metallization (UBM). The solder balls used in conventional flip-chip bonding processes are usually bigger than 100 μm, and thus, are not suitable for fine pitch interconnections (e.g., less than 100 μm), such as, for example, those used in the recording interface described above, which are on the order of 75 μm. Accordingly, rather than using solder, the method 100 uses ACF to bond together components of the recording interface.

ACF has a different bonding mechanism than solder—thermosetting of resins embedded with metal-coated conductive spheres. Compared with solder reflow used in conventional flip-chip bonding, ACF intrinsically shows far less electrical short probability because conductive sphere particles stay insulated without pressure. Another advantage of ACF flip-chip bonding over conventional flip-chip bonding with solder reflow is that ACF is readily under-filled with non-conductive, spreadable resins, and thus, no additional under-filling is required. It also does not need post-CMOS patterning/metallization on the pads of the integrated circuit since no melting/reflow of metal is included in the process.

In any event, in a first step 102 of the method 100, ACF is deposited onto the top surface of the integrated circuit 14 at the locations of the area pads 42 to be electrically connected to and bonded with the bumps 40 of the probe terminal/backend 30 (e.g., pads 42 having active circuits 44 buried thereunder). In an embodiment, this comprises depositing discrete balls, spheres, or bumps of ACF on each pad 42 of the integrated circuit 14 that is to be coupled with a bump 40 of the neural probe terminal 30. In an embodiment, the ACF used has a double layer structure composed of an ACF layer and a non-conducting film (NCF) layer. This type of ACF is used because the resin in the ACF layer has high viscosity so that it can contain conductive particles during the bonding process, and the NCF has low viscosity so that it can provide fluidity of the film to make good mechanical connections/bonds. One suitable ACF is that supplied by Hitachi Chemical under product number AC-8955YW. While a particular type of ACF has been described above, it will be appreciated that other suitable types of ACF may be used, and thus, the present disclosure is not limited to any particular type(s) of ACF.

In a second step 104, the probe terminal 30 is aligned with the integrated circuit 14 such that the bumps 40 projecting from the bottom surface 30 of the terminal 30 are aligned with respective pads 42 of or on the top surface of the integrated circuit 14. The probe terminal 30 and integrated circuit 14 are then pressed together in a step 106 such that each bump 40 of the probe terminal 30 contacts the ACF ball 52 at a respective area pad 42 location of the integrated circuit 14. In an embodiment, the terminal 30 and the integrated circuit 14 are pressed together with an applied pressure of around 80 MPa, and at a temperature of around 150-230°; though other pressures and/or temperatures may certainly be used instead. Finally, in step 108, the combination of the terminal 30 and the integrated circuit is cooled to allow the ACF to harden or cure. In an embodiment, the cooling is carried out by a nitrogen purge. One or more of steps 104, 106, and 108 described above may be carried out using any suitable flip-chip bonding tool. One example of such a tool is the FINEPLACER® lambda manufactured by Finetech GmbH & Co. KG located in Berlin, Germany, though other suitable tools may certainly be used instead.

In an embodiment, the flip-chip bonding process described above is used to bond and electrically connect the bumps 40 of the probe 12 with the pads 42 and active circuits 44 of the integrated circuit 14. Other pads of the integrated circuit not having an active circuit buried thereunder, for example, the pads 54 for the connection of power, ground, and data communication may be connected using a conventional wire-bonding technique.

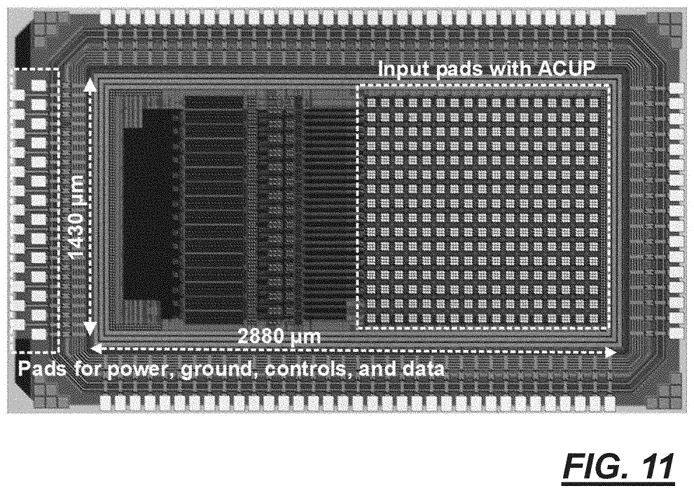

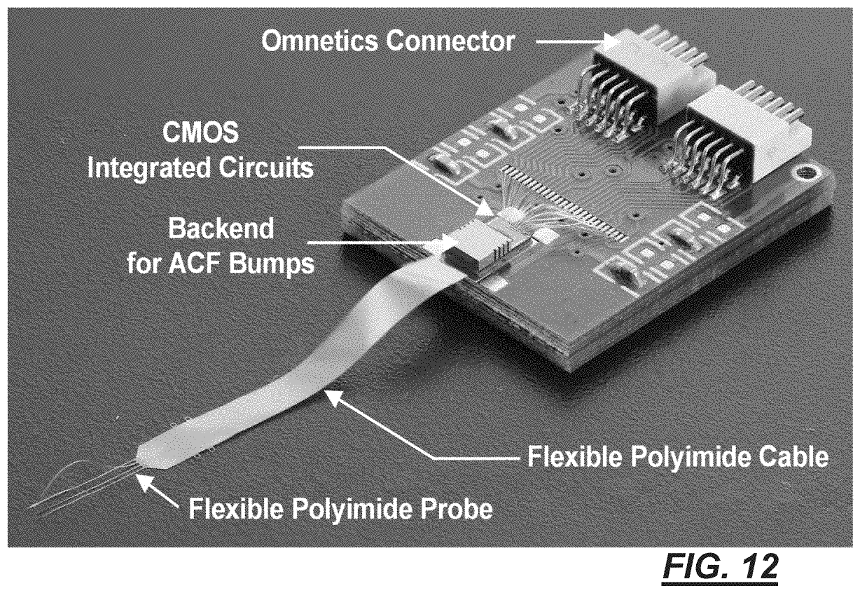

To demonstrate the feasibility and functionality of both the recording interface 10 and integrated circuit 14 described above, a prototype recording front-end integrated circuit was fabricated using 180 nm 1P6M CMOS processes. FIG. 11 shows a microphotograph of the fabricated chip. The active area was realized in 2.88 mm²×1.43 mm^{2,} excluding wire-bonding pads. The top, bottom, and right pads are for test and characterization of circuit functionality. The left pads are allocated for providing power, ground, and digital control and output signals (SPI bus). The area pads (an array of 16×17) for connection to recording and reference electrodes are located inside the dashed-line box on the right side of the chip. The fabricated chip was hybrid-assembled with a polyimide neural probe using ACF bonding. The ACF bonding process was conducted using a flip-chip bonder that enables alignment tolerance of less than 3 μm. A small pierce of ACF (2 mm×3 mm) was pre-bonded on the chip after removing a protective film. Then, the protective film on the other side was removed and the probe backend (i.e., the probe terminal) was pre-bonded on the ACF layer by the flip-chip bonder. The probe/ACF/integrated circuit stack was pressed in the same tool at 80 MPa with a chuck temperature of 230° C. The integrated circuit was then mounted on a PCB with glue and silver paste. Finally, the pads of the integrated circuit for power, ground, and SPI signals from the integrated circuit components were wire-bonded to the PCB which has two (2) 12-pin Omnetics connectors. FIG. 12 shows an assembled minimally-invasive 256-channel neural recording interface.

During benchtop testing, the measured differential mode (DM) and common mode

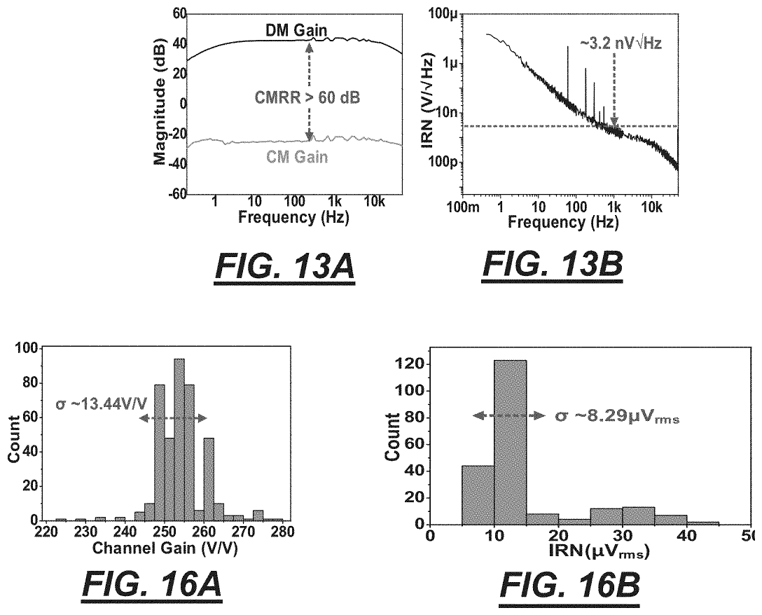

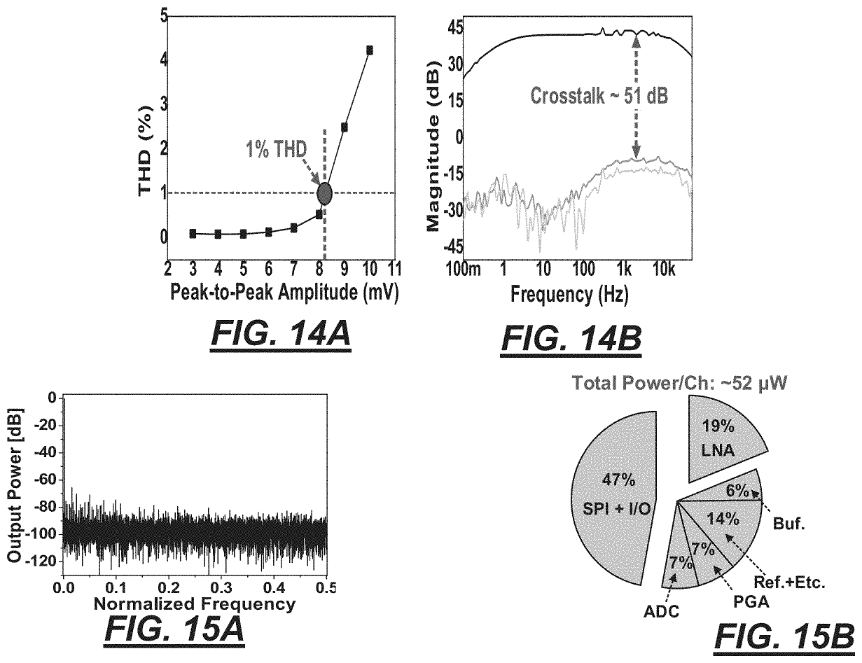

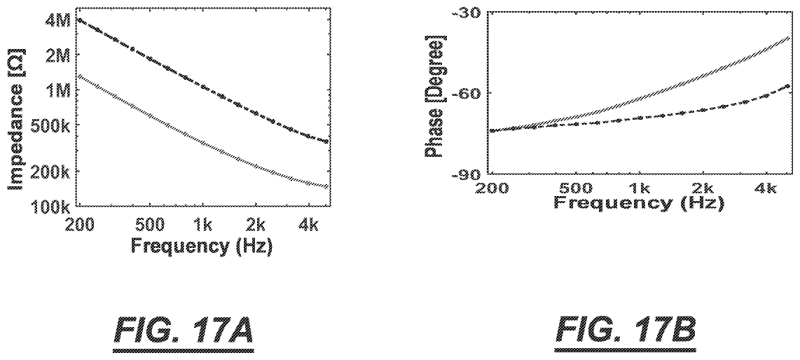

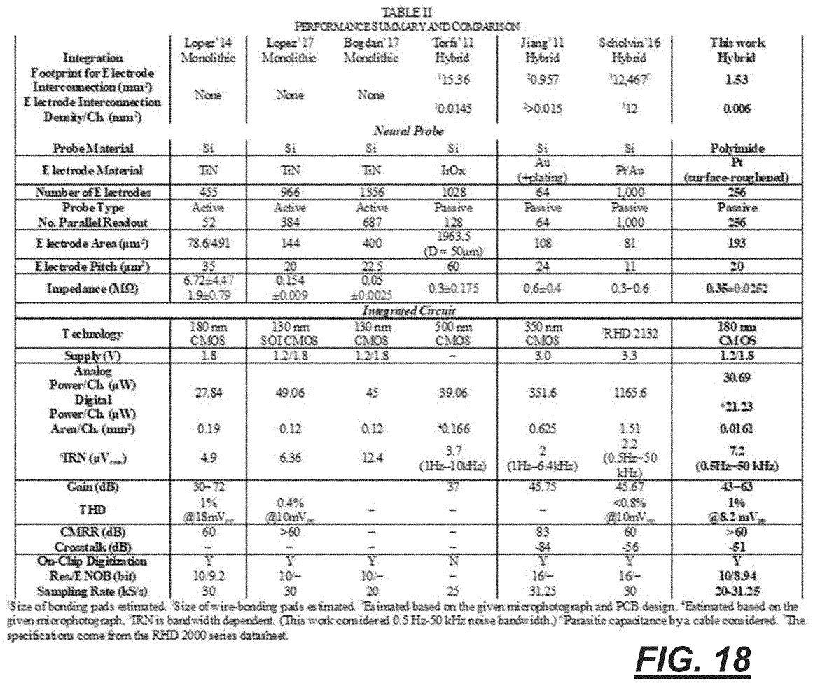

(CM) gains of the amplifier of an active pixel buried under an area pad of the integrated circuit, which in the prototype comprised an LNA, are shown in FIG. 13A. The LNA has a mid-band gain of ˜43 dB from ˜1.7 Hz (f_{L}) to ˜17.5 kHz (f_{H}), and gives a CMRR of ˜60 dB in the mid-band. Because of process variation in the pseudo-resistor and leakage current in the T-network comprising the feedback capacitance C_{fb}, f_{L} was measured off from the designed value (less than 0.4 Hz). The measured IRN of the LNA is shown in FIG. 13B. The integrated IRN from 0.5 Hz to 50 kHz was measured to be ˜7.2 μVᵣₘₛ, satisfying the noise requirement for neural recording (less than 10 μVᵣₘ). The 1/f noise corner frequency was relatively high (estimated at ˜3 kHz) due to the small input transistors but the thermal noise floor was found to be smaller than the designed-for value of ˜3.2 nV√Hz at 1 kHz. The total harmonic distorting (THD) of the LNA from 1 kHz sine wave input was measured as shown in FIG. 14A. Until applying up to 8.2 mVₚₚ input, the THD was suppressed below 1%. The LNA showed a relatively large input range owing to the reference-replica scheme (wherein the non-inverting input of the LNA is electrically connected to the non-inverting input of the amplifier (e.g., LNA) of a reference pixel, which, in turn, is electrically connected to a common voltage) where four (4) input transistors take the input signals. FIG. 14B shows the worst-case crosstalk of −51 dB among 6 channels. The power consumption of the LNA was measured as ˜8.57 μW. Based on the foregoing measurements, the performance metrics NEF and NEF²V_{DD} were calculated to be 6.11 and 44.79, respectively. FIG. 15A shows the 16,348 points of FFT results in the SAR ADC from 1 kHz sine wave input. Based on the FFT, SNDR was estimated as ˜55.61 dB and the effective number of bit (ENOB) was calculated to be 8.944 bits. FIG. 15B depicts the percentile of the measured power consumption per channel. The analog and digital power consumptions per channel were 31.56 and 21.23 μW, respectively, including power consumption to drive parasitic capacitance in the PCB. The total power consumption for the entire integrated circuit chip from analog 1.2 V and digital 1.8 V supplies were measured as ˜8.08 and ˜5.44 mW, respectively, including current/voltage reference circuits and digital control circuits, such as ADC controllers, registers, and 8 SPIs. FIGS. 16A and 16B show the gain variation and noise distribution across the entire 256 channels, measured from an in vitro test setup using phosphate buffered saline solution (PBS). The overall system yield, including the probe, integrated circuit, and assembly yields, was 83.2% in the in vitro measurement set up (213 out of 256 channels were properly working). The noise variation not only includes the whole signal processing blocks, such as the LNA, PGA, and ADC, but also combines the electrode noise and environment interference (50 or 60 Hz noise and other coupled interference). The mean and the standard deviation of gain were found to be ˜227.45 and ˜13.44 V/V (˜6%), respectively, mostly coming from impedance variation in the electrodes that had been fabricated using the in-house MEMS processes (˜7%). The mean and standard deviation of IRN were found to be ˜14.86 and ˜8.69 μVᵣₘₛ, respectively. The additional noise may come from the electrode impedance (spot noise: ˜76 nV√Hz) and interference, such as 50 or 60 Hz noise, and inductively coupled noise in the measurement lines in the in vitro setup. The impendence of the electrodes, which comprised surface-roughened platinum electrodes, was measured in a PBS between 200 Hz and 5 kHz using nanoZ impedance tester, as shown in FIG. 17A. The measured impedance of the electrodes with an area of 193 μm² was 347.6 kΩ at 1 kHz in average over 101 electrodes, which, as shown in FIG. 17A, is three (3) times smaller than that of plain platinum electrode impedance (1.055 MΩ) with an identical area. The table comprising FIG. 18 compares the performance with other known recording interfaces.

During in vivo testing, protocols and procedures approved by the Institutional Animal

Care and Use Committee of the New York University and the University of Michigan IACUC were followed. Male Long Evans rats (380 g) were used in the acute study. The rats had been kept on a regular 12-hour light/12-hour dark cycle and housed in pairs before surgery. No prior experimentation had been performed on the animals. Atropine (0.05 mg/kg, s.c.) was administered after isoflurane anesthesia induction required to reduce saliva production. The body temperature was monitored and kept constant at 36-37° C. with a DC temperature controller (TCAT-LV; Physitemp, Clifton, N.J.). Stages of anesthesia were maintained by confirming the lack of nociceptive reflex. Skin of the head was shaved and the surface of the skull was cleaned with hydrogen peroxide (2%). A ground screw was positioned above the cerebellum. Then, a 1.8 mm diameter craniotomy was drilled at 3 mm posterior from bregma and 2 mm lateral from the midline. The dura was removed and the flexible probe was attached on a shuttle and lowered a one (1) mm/min speed until characteristic electrophysiological features of the CA1 region of the hippocampus were observed on the channels located at the tip of the probe. To test an active probe, the craniotomy was filled with sterile saline solution and two (2) hours recording was performed under anesthesia. The recorded signals (n=256) were acquired using C code-based recording software. The recorded data was analyzed by custom scripts written in MATLAB (MathWorks, USE). Offline spikes were detected and automatically sorted using the Kilosort algorithm followed by manual curation using Phy to get well-isolated single units.

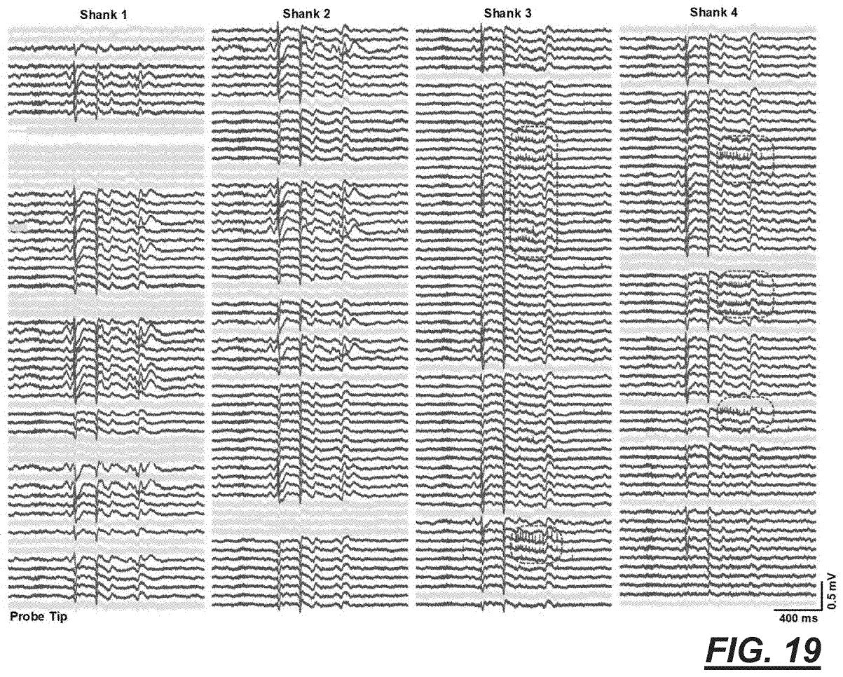

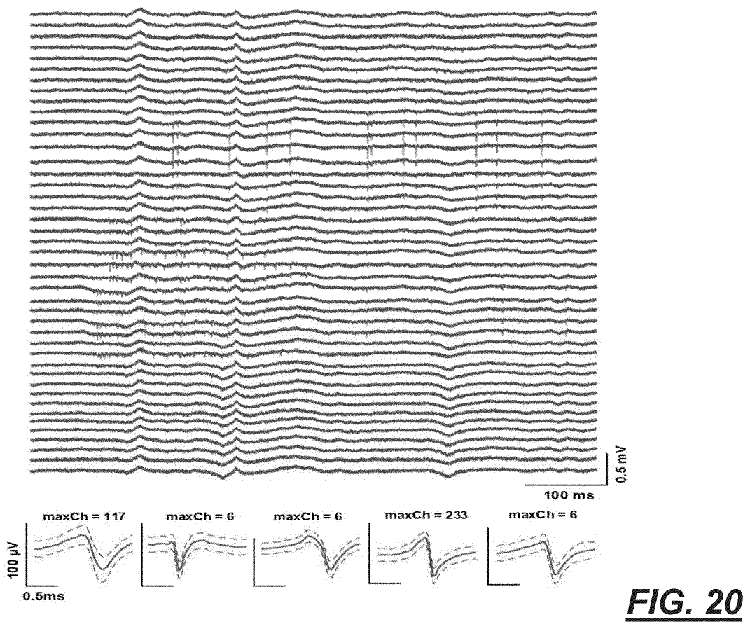

The recorded in vivo signals from 256 channels are shown in FIG. 19. These unfiltered, raw traces for the four shanks which are 250 μm apart from each other. Clear local field potential (LFP) signals such as sharp ripples were successfully recorded from a total of 210 channels (˜82%). The channels with a low SNR of less than 30 dB were marked. Some spike events are indicated with a dashed or dotted box showing firing from several neighboring channels and gradual distribution in amplitude along those channels. After spike sorting of 25-minute recording session data, 20 clean clusters were found by automatic sorting and manual curation. Some examples of the detected single units are displayed in FIG. 20. The calculated RMS noise after high pass filtered at 500 Hz was 17 μVᵣₘₛ. Through the in vivo experiments, the functionality of the assembled package was evaluated by the recorded neural LFP and spike gains.

It is to be understood that the foregoing description is of one or more embodiments of the invention. The invention is not limited to the particular embodiment(s) disclosed herein, but rather is defined solely by the claims below. Furthermore, the statements contained in the foregoing description relate to the disclosed embodiment(s) and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art.

As used in this specification and claims, the terms “e.g.,” “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Further, the term “electrically connected,”, “electrically coupled,” and the variations thereof are intended to encompass both wireless electrical connections and electrical connections made via one or more wires, cables, or conductors (wired connections), as well as direct electrical connections between two or more components and indirect connections wherein one or more components are connected or coupled between to components that are electrically connected or electrically coupled together. Other terms are to be construed using their broadest reasonable meaning unless they are used in a context that requires a different interpretation. In addition, the term “and/or” is to be construed as an inclusive OR. Therefore, for example, the phrase “A, B, and/or C” is to be interpreted as covering all the following: “A”; “B”; “C”; “A and B”; “A and C”; “B and C”; and “A, B, and C.”

1. A neural recording interface, comprising:
a neural probe including:
an elongate cable having a first end and a second end;
an implantable portion at the first end of the cable and including plurality of electrodes; and
a terminal at the second end of the cable having a body with a top surface and a bottom surface opposite the top surface, and a plurality of bumps projecting from the bottom surface of the terminal body, wherein each bump is electrically connected to a corresponding one of the plurality of electrodes; and
a recording integrated circuit having:
a plurality of area pads; and
a plurality of active circuits each disposed under a respective one of the plurality of area pads;
wherein each of the plurality of bumps projecting from the bottom surface of the probe terminal body is bonded to a respective one of the plurality of area pads of the integrated circuit with an anisotropic conductive film such that each of the plurality of electrodes of the neural probe is electrically connected to a respective one of the active circuits of the integrated circuit.

2. The neural recording interface of claim 1, wherein the plurality of active circuits of the integrated circuit comprises a plurality of active pixels.

3. The neural recording interface of claim 2, wherein each of the plurality of the active pixels comprises:
an amplifier having an inverting input, a non-inverting input, and an output;
a coupling capacitor electrically connected to the inverting input of the amplifier and coupling the amplifier with the electrode of the neural probe to which the active pixel is electrically connected;
a feedback capacitor electrically connected between the inverting input and the output of the amplifier; and
a resistor electrically connected in parallel with the feedback capacitor.

4. The neural recording interface of claim 3, wherein the amplifier comprises a low noise amplifier (LNA).

5. The neural recording interface of claim 4, wherein the LNA comprises an operational transconductance amplifier.

6. The neural recording interface of claim 3, wherein a first active pixel of the plurality of active pixels comprises an active recording pixel and the electrode to which it is electrically connected comprises a recording electrode, and a second active pixel of the plurality of active pixels comprises an active reference pixel and the electrode to which it is electrically connected comprises a reference electrode, and further wherein the non-inverting inputs of the amplifiers of the first and second active pixels are electrically connected together and to a common voltage.

7. The neural recording interface of claim 6, wherein a third active pixel of the plurality of active pixels comprises an active recording pixel and the electrode to which it is electrically connected comprises a recording electrode, and further wherein the non-inverting inputs of the amplifiers of the first, second, and third active pixels are electrically connected together and to the common voltage.

8. The neural recording interface of claim 3, wherein the integrated circuit further includes a programmable gain amplifier (PGA), and the output of each of the amplifiers of the plurality of active pixels is electrically connected to an input of the PGA.

9. The neural recording interface of claim 8, wherein the PGA comprises a fully differential amplifier.

10. The neural recording interface of claim 8, wherein the integrated circuit further includes a multiplexer having a plurality of inputs and an output, and further wherein the outputs of the amplifiers of the plurality of active pixels are electrically connected to respective inputs of the multiplexer, and the output of the multiplexer is electrically connected to an input of the PGA.

11. The neural recording interface of claim 8, wherein the integrated circuit further includes an analog-to-digital converter (ADC), and further wherein an output of the PGA is electrically connected to an input of the ADC.

12. The neural recording interface of claim 11, wherein the integrated circuit further includes a series-parallel interface (SPI) having an input and an output, and further wherein an output of the ADC is electrically connected to the input of the SPI.

13. The neural recording interface of claim 3, wherein the coupling capacitor comprises a metal-insulator-metal capacitor formed in part from a first metal layer and a second metal layer of the integrated circuit.

14. The neural recording interface of claim 13, wherein the first metal layer comprises a top metal layer of the integrated circuit, and further wherein the bumps of the neural probe terminal are bonded to respective portions of the first metal layer by the anisotropic conductive film.

15. The neural recording interface of claim 1, wherein the neural probe includes at least 256 electrodes, and the integrated circuit chip includes at least 256 area pads and active circuits.

16. The neural recording interface of claim 1, wherein each of the bumps of the terminal of the neural probe has a height of 15-20 μm.

17. The neural recording interface of claim 1, wherein each of the active circuits of the integrated circuit has an area of 75 μm.

18. A neural recording integrated circuit configured for use with a neural probe, comprising:
a plurality of active pixel circuits, wherein each active pixel circuit includes:
an amplifier having an inverting input, a non-inverting input, and an output;
a coupling capacitor electrically connected to the inverting input of the amplifier and configured to electrically couple the amplifier with a respective electrode of the neural probe when the integrated circuit is electrically coupled with the neural probe;
a feedback capacitor electrically connected between the inverting input and the output of the amplifier; and
a resistor electrically connected in parallel with the feedback capacitor.

19. The integrated circuit of claim 18, wherein the amplifier comprises a low noise amplifier (LNA).

20. The integrated circuit of claim 19, wherein the LNA comprises an operational transconductance amplifier.

21. The integrated circuit of claim 18, wherein a first active pixel of the plurality of active pixels comprises an active recording pixel configured to be electrically connected to a recording electrode of the neural probe, and a second active pixel of the plurality of active pixels comprises an active reference pixel configured to be electrically connected to a reference electrode of the neural probe, and further wherein the non-inverting inputs of the amplifiers of the first and second active pixels are electrically connected together and configured to be electrically connected to a common voltage.

22. The integrated circuit of claim 21, wherein a third active pixel of the plurality of active pixels comprises an active recording pixel configured to be electrically connected to another recording electrode, and further wherein the non-inverting inputs of the amplifiers of the first, second, and third active pixels are all electrically connected together and configured to be electrically connected to the common voltage.

23. The integrated circuit of claim 18, further comprising a programmable gain amplifier (PGA), and the output of the amplifier of each of the active pixels is electrically connected to an input of the PGA.

24. The integrated circuit of claim 23, wherein the PGA comprises a fully differential amplifier.

25. The integrated circuit of claim 23, further comprising a multiplexer having a plurality of inputs and an output, and further wherein the output of the amplifier of each of the plurality of active pixels is electrically connected to a respective input of the multiplexer, and the output of the multiplexer is electrically connected to an input of the PGA.

26. The integrated circuit of claim 23, further comprising an analog-to-digital converter (ADC), and further wherein an output of the PGA is electrically connected to an input of the ADC.

27. The integrated circuit of claim 26, further comprising a series-parallel interface (SPI) having an input and an output, and further wherein an output of the ADC is electrically connected to the input of the SPI.

28. The integrated circuit of claim 18, wherein the coupling capacitor comprises a metal-insulator-metal capacitor formed in part from a first metal layer and a second metal layer of the integrated circuit.

29. The integrated circuit of claim 18, wherein each active pixel has an area of 75 μm.

30. A method of assembling a neural recording probe having a terminal with a plurality of bumps projecting from a bottom surface thereof, with a neural recording front-end integrated circuit having a plurality of area pads each having an active circuit buried thereunder, comprising:
depositing an anisotropic conductive film (ACF) onto area pads of the integrated circuit;
aligning a terminal of the neural probe with the integrated circuit such that the bumps projecting from the bottom surface of the terminal are aligned with respective area pads of the integrated circuit;
applying pressure between the terminal and the integrated circuit at a predetermined temperature; and
cooling the terminal and the integrated circuit until the ACF hardens.
